(19) United States
(12) Patent Application Publication
(10) Pub. No.: US 2007/0161779 A1
(43) Pub. Date: Jul. 12, 2007

(54) Estrogen Extraction From Pregnant Animal Urine

(75) Inventor: Joseph A. Vella, Melbourne (AU)

Correspondence Address: CONNOLLY BOVE LODGE & HUTZ LLP, P.O. BOX 2207, WILMINGTON, DE, 19899-2207, US

(73) Assignee: Thorgard Pharmaceuticals (AUST) Pty Ltd, Melbourne (AU)

(21) Appl. No.: 10/576,726

(22) PCT Filed: Oct. 25, 2004
(86) PCT No.: PCT/AU04/01454

§ 371 Date: Oct. 10, 2006

(30) Foreign Application Priority Data
Oct. 24, 2003 (AU) 2003905834

(51) Int. Cl. C07J 1/00

(52) U.S. Cl. 530/326; 552/625; 530/398

(57) ABSTRACT

A method of extracting oestrogens, conjugated estrogens, equine gonadotropin and equine follicle stimulant from pregnant mares urine which includes the steps of contacting the urine with dispersed particles of adsorber materials of a particle size above 200 microns and subsequently removing the species by washing the adsorber materials in a buffered aqueous solution of pH between 7.5 and 9.5. The compounds are extracted in situ on the mare using an apparatus which consists of a urine holding vessel suspended beneath the belly of the mare and a urine collection device attached to the perineurn of the mare and communicating with the holding vessel in which the urine collection device and/or the holding vessel incorporates a removable urine permeable container of an adsorbent material for said chemical species. Alternatively the holding container can contain a water immiscible solvent such as hexanol for the in situ extraction of estrogens. An initial filter is incorporated into the urine collection device upstream of the adsorber container. The filter removes mucous and other viscous component of the urine. The system is also applicable to cattle.

This invention relates to a device and a method of extracting estrogens and valuable proteins and peptides from pregnant animal's urine.

### BACKGROUND OF THE INVENTION

Pregnant mare's urine (PMU) is valued as a source of hormones for use in hormone replacement therapy.

The collection or harvesting of the urine can be achieved using collection apparatus that is carried by a harness below the horses belly and fits about the mares perineurn. Apparatus of this kind is disclosed in Australian patents 725205, 743611, 748458 and 763340.

The collected urine is treated to remove the hormones. U.S. Pat. No. 5,723,454 discloses a method of solid phase extraction in which the estrogens are collected on a semi-polar polymeric resin and stripped from the resin by a first step of washing at a pH above 12 and the removing the hormones with a water miscible organic solvent.

U.S. Pat. No. 5,814,624 discloses a method of treating the urine with a water soluble base at a pH above 12 and then contacting the treated urine with a hydrophobised silica gel followed by desorption with a water miscible organic solvent.

WO 01/27134 discloses a method of extraction in which the PMU is contacted with a salt followed by liquid/liquid extraction.

USA application 2002/0156303 discloses a method of treating the PMU with an alkaline solvent, followed by filtering and then by contact with polystyrene absorber resins and removing the hormones by eluting with a water miscible organic solvent.

USA patent application 2003/0105344 discloses a method of using adsorber cartridges to enable the estrogens to be adsorbed in the cartridges on the farm so that the urine need not be transported off the farm. The cartridges can be taken to a central stripping facility and returned to the farm for reuse.

Urine from pregnant mares is not easily handled because it is often viscous and may contain a high proportion (up to 40% in some cases) of mucous and mucosal cells; a high level of nitrogenous compounds predominantly urea and ureides; proteins and protein degradation products; as well as potentially high microbiological loads of bacteria and some fungal species. The current procedures can be costly requiring sophisticated equipment and generate waste residues which may not be easily disposed of in a non farm environment.

It is an object of this invention to overcome these problems and to provide a convenient means of extracting hormones and other valuable biological compounds from PMU.

### BRIEF DESCRIPTION OF THE INVENTION

To this end the present invention provides apparatus for the collection of valuable chemical species from pregnant animal's urine which consists of a urine holding vessel suspended beneath the belly of the animal and a urine collection device attached to the perineum of the pregnant animal and communicating with the holding vessel in which the urine collection device and/or the holding vessel incorporates a removable urine permeable container of an adsorbent material for said chemical species.

Alternatively the holding vessel can simply contain a non water miscible organic solvent for the desired chemical species. A preferred solvent is hexanol which may be placed in the holding vessel and when the urine enters the chamber the motion of the animal will mix the two phases and allow estrogens to be dissolved in the hexanol phase. The two phases can be emptied from the holding vessel and separated. The solvent can be evaporated to leave a dry estrogen rich powder. The treated urine may be extracted with hexanol a second time to improve the yield.

By incorporating a removable adsorbent container in the urine collection device or the holding vessel the urine may be discarded on site. Depending on the extraction efficiency the urine may be allowed to drain from the holding vessel while on the mare thus avoiding any disposal problems. The adsorbent container can be transported to a central treatment facility for removal of the desired chemical species and the container may be returned for reuse.

An initial filter may be incorporated into the urine collection device upstream of the adsorber container. This filter may be dosed with a mucolytic agent such as sputolisinto remove mucous and mucosal cells and reduce viscosity.

Additional filters or mucolytic agent carriers may be incorporated in the collection device to complete the initial treatment and reduce fouling of the adsorber materials.

The adsorber material may be presented in a flow-through cartridge or a loose filled porous container within the urine holding container. The movement of the animal will ensure that the urine contacts the adsorber material. If desired the urine can be treated a second time to increase yield of the desired chemical species. This could be done by collecting the urine and passing it in the conventional manner through a packed column. Preferably the urine is passed through cartridges of the collector material. Alternatively the urine can be allowed to slowly flow out of the holding container and a second cartridge or container of adsorber material can be located at the outlet to effect a second collection.

It is within the scope of this invention to include within the holding container a solvent container to be used for liquid-liquid extraction of the desired chemical species.

The system and method of this invention is applicable to any farm animal that provides a source of valuable chemical's in its urine. Mares are an established source of estrogen but the system is applicable to cattle and other farm animals.

The chemical species to be targeted are oestrogens, conjugated estrogens, equine chorionic gonadotropin, follicle stimulating hormone (FSH) and other biological proteins and peptides.

The adsorber materials may be selected from any known polymeric adsorbers or silica-based absorbers. These include ion exchange resins or non ionic semipolar resins such as acrylic resins, Agarose and crosslinked agarose resins, silica-based adsorbents with bonded ligands as used in hydrophobic interaction media. To accommodate the flow requirements in the collection device the particle size of the adsorbents is above 200 microns and preferably greater than 500 microns.

The chemical species may recovered by any suitable method including using a water miscible organic solvent as eluant. It is preferred to use a buffered aqueous solution of pH 7.5-9.5 to recover the compounds. The solution may be desalted and then dried to produce a powder of the chemical species.

### DETAILED DESCRIPTION OF THE INVENTION

Preferred embodiments of the invention will be described with reference to the drawings in which:

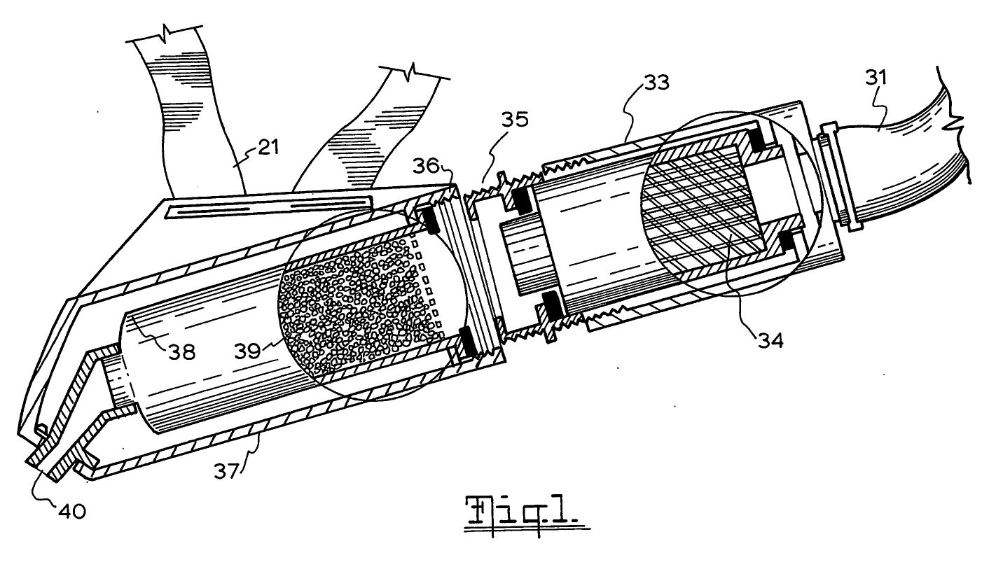

FIG. 1 illustrates a urine collection device according to a first embodiment of this invention;

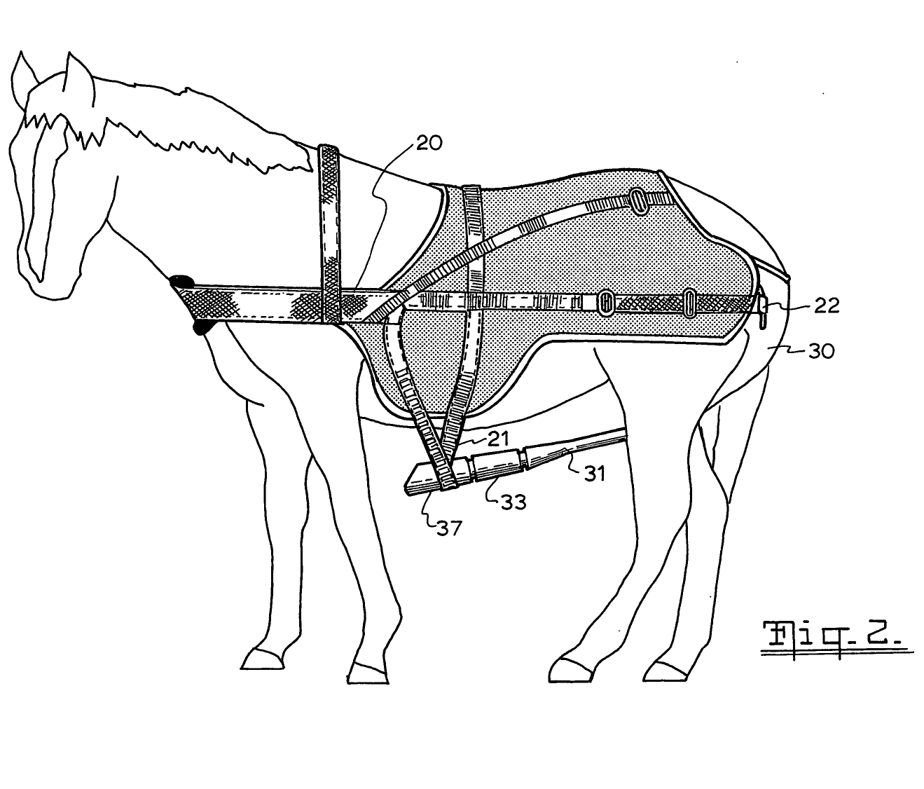

FIG. 2 illustrates the embodiment of FIG. 1 attached to a mare;

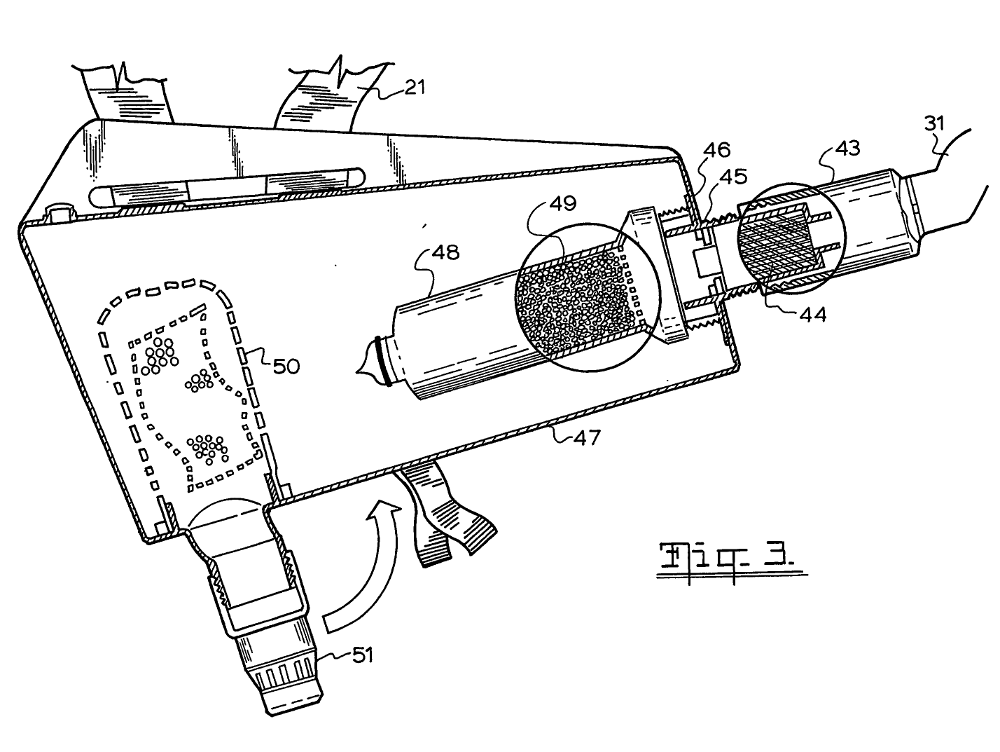

FIG. 3 illustrates a urine collection device according to a second embodiment of this invention;

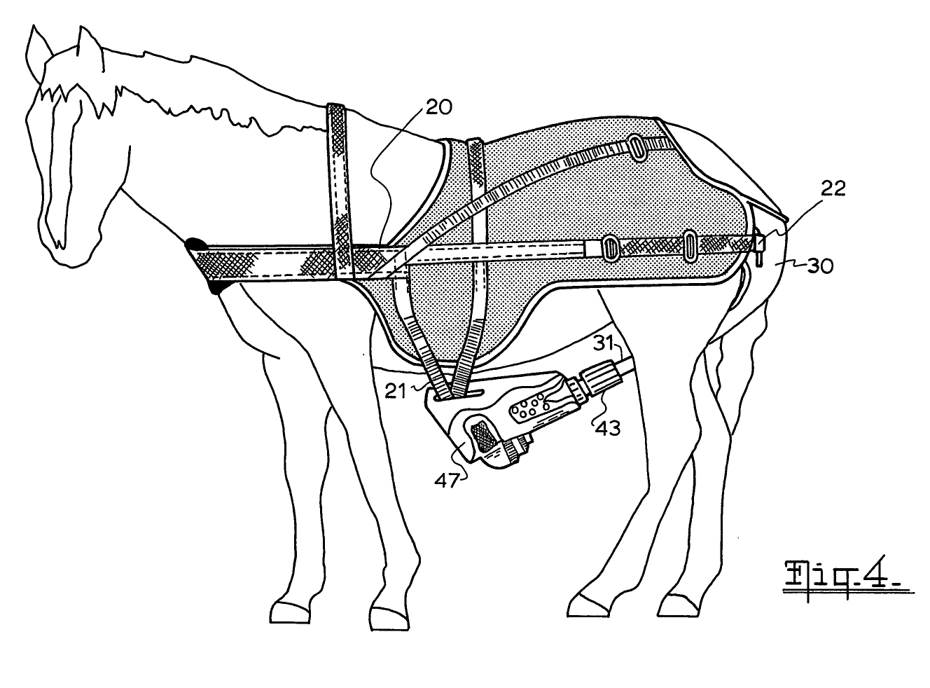

FIG. 4 illustrates the embodiment of FIG. 3 attached to a mare;

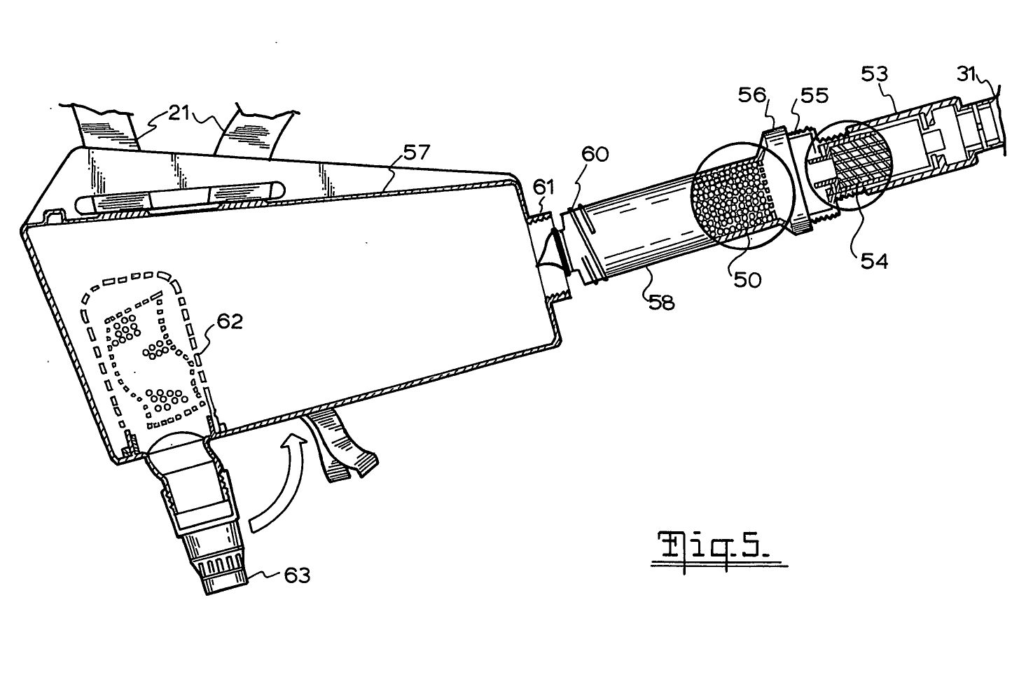

FIG. 5 illustrates a urine collection device according to a first embodiment of this invention;

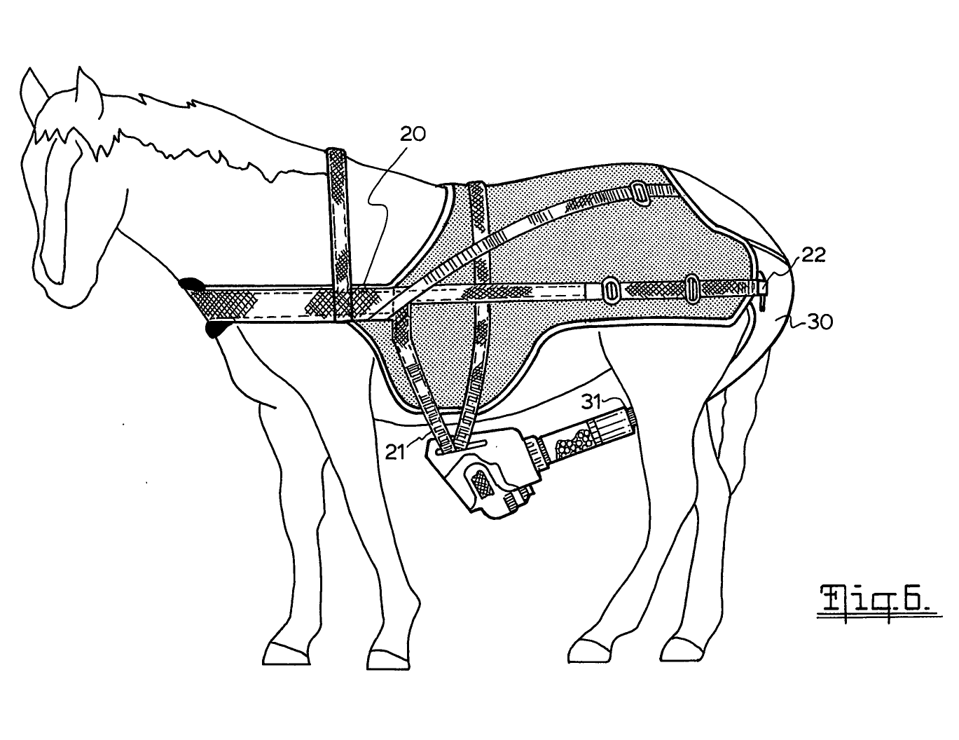

FIG. 6 illustrates the embodiment of FIG. 5 attached to a mare.

The apparatus is based on the apparatus disclosed in Australian patent 748458 and is modified to accommodate the filters and adsorption cartridges as taught by this invention. The contents of patent 748458 are incorporated herein by reference.

The first embodiment shown in FIGS. 1 and 2 is a simple flow through arrangement where the urine is allowed to flow through to the ground. In all the embodiments the urine collector is mounted on the mare by harness 20. the straps 21 hold the main urine receptacle and the straps 22 hold the perineum engagement means 30 into which the mare urinates. The urine flows into the collection tube 31 which passes between the mares hindlegs towards its underbelly.

The tube 31 preferably incorporates a filter (not shown) which is a funnel shaped high flow rate filter and may incorporate a mucolytic agent to reduce the viscosity of the urine. The filter in tube 31 is funnel shaped. This filter may also be incorporated in the perineum engagement means 30 adjacent the urine entry point. This filter may also be funnel shaped and be located just below the mare's vulva. The preferred filter material is a polyamide mesh with apertures of 300 microns and filaments of 215 microns. Polyester or polypropylene meshes may also be used and depending on the urine different mesh sizes may also be used. Meshes marketed by Sefar Inc Filtration division of Switzerland are preferred. This filter may also incorporate a coarse (up to 2 mm) particular adsorbent such as one or more of perlite, or vermiculite attapulgite, zeolite to assist in the entrapment of cellular debris and to bind mucosal cells and/or nitrogenous compounds.

The filter unit 33 incorporates a urine filter 34 containing mucolytic agent such as sputolisin. The filter unit 33 is located upstream of the urine receptacle 37. The filter unit 33 includes a screw threaded or snap fastening connector 35 which interfits with a complementary connector 36 of the receptacle 37. The volume of the collector filter unit 33 may need to be increased to take account of the volume lost by insertion of the filter 34 and the reduced flow rate due to the filter 34. Again the filter material is preferably a polyamide mesh with apertures of 300 microns and filaments of 215 microns. If a filter unit is located adjacent the perineum in the tube 30 the second filter unit 33 may not be necessary.

The receptacle 37 includes a flow through collector unit 38 incorporating collector beads 39 for the conjugated estrogens. In this embodiment the urine flows over the beads 39 and then through the outlet 40 on to the ground. The adsorber materials may be selected from cellulose based resins, polyacrylamide based resins, acrylic resins such as Amberlite AG2X8, Amberlite IRA96, Amberlite FPA51, Amberjet 4200Cl, Amberjet 4400Cl or equivalent resins, crosslinked Agarose, silica adsorbents with bonded ligands such as C-2, C-8, or C-18 side chains. A mixture of a weak base anionic resin such as Amberlite FPA51 and a strong base anionic resin such as Amberjet 4400Cl is preferred. ( Amberlite and Amber jet are trademarks of Rohm & Haas.)

Larger bead absorbents may also be used which need not be in a column but which can be used loose in a wide bed. These include Big Beads by Amersham and beads sold by BioRad or a matrix with a titanium core. Primarily oestrogens or conjugated estrogens with bonded sugar groups are extracted. But other active biological agents such as equine chorionic gonadotropin and follicle stimulating hormone may be collected. These compounds need to be kept in the aqueous phase to avoid denaturing.

In the embodiment of FIGS. 1 and 2 the residence time of the urine on the collector beads is relatively short and the yield of desirable chemical species may not be high.

The embodiment of FIGS. 3 and 4 provides a larger holding receptacle for the urine. As in the first embodiment a filter unit 43 with filter 44 similar to that in the first embodiment is positioned upstream of the collection receptacle 47. Again connection means 45 and 46 allow the filter unit 43 to be connected to the receptacle unit 47. A cartridge 48 of adsorber particles 49 (similar to those used in the first embodiment) is placed in the inlet to the container 47 so that the urine flows through or around the cartridge 48. The cartridge 48 should be porous and may be a perforated cylinder or a sachet of porous material similar to that used for tea bags such as the non woven cellulose and polypropylene paper sold by Dexter Corporation.

The volume of the receptacle 47 is large enough to retain 24 hours urine discharge from the mare so that the residence time of the beads 49 in the urine can be several hours until the receptacle is drained. The holding container 47 may have a flow controlling valve at the inlet to allow the urine to gently flow over the adsorber materials 49 and 50 which may be immersed in the holding container in loose porous bags or be suspended as a cylindrical cartridge 48 for flow through extraction.

When the outlet valve 51 is opened the urine may be discharged into the ground or into another collection vat for additional processing. Adjacent the outlet 51 is a second collector unit that collects the desired chemical species remaining after the urine flows through unit 48. The collector unit 50 also executes a final collection as the urine flows over it to exit through outlet 51. The valve 51 can be adjusted to allow slow release of the urine from the holding chamber to increase the contact time with the adsorber materials. In the third embodiment shown in FIGS. 5 and 6 the urine capacity of the receptacle 57 is increased by locating the flow through collection cartridge 58 outside of the container 57. the urine flows through the collection tube 31 into the filter unit 53 containing a filter 54 (of the same type as in the first two embodiments). Again the connectors 55 and 56 allow easy connection of the filter unit 53 to the collection cartridge 58. Similar connection means 60 allow the cartridge 58 to be easily connected at 61 to the inlet of the receptacle 57.

Adjacent the outlet 63 is a second collector unit 62 that collects the desired chemical species remaining after the urine flows through unit 58. This will be dependent on the residence time of the urine in receptacle 57. The collector unit 62 also executes a final collection as the urine flows over it to exit through outlet 63.The valve 63 can be adjusted to allow slow release of the urine from the holding chamber to increase the contact time with the adsorber materials.

The adsorber materials 59 and 62 are the same as in the first two embodiments.

The holding containers 37, 47 and 57 in all 3 embodiments is designed so that the filters and adsorber cartridges can easily be removed and replaced by fresh filters and adsorbers. The saturated adsorbers may be transported to a central extraction facility where they are treated to remove the estrogens etc and the adsorber materials are regenerated. This stripping may be achieved using water miscible solvents such as methanol, ethanol, hexanol, ketone etc. Preferably an aqueous solvent is used which is a buffered solution with a pH in the range of 7.5 to 9.5. The solution may contain bicarbonate or phosphate ions as the buffer and concentrations of a salt. The regenerated beads can be reused in the urine collection devices.

In another form the invention provides a suitable urine receptacle container (not shown) large enough to accommodate an extraction solvent hexanol. This is a known liquid liquid extraction technique for dissolving estrogens in the hexanol which is a separate phase to the urine. The motion of the horse ensures that the extraction is complete by the time the fluids are drained from the holding container. This is usually effective in extracting 90% of the estrogens and a second extraction in a separate vat can increase the yield to 99%.

Those skilled in the art will realize that the invention provides a convenient means of avoiding unnecessary transport of urine without sacrificing yield. Other advantages of this method are that individual animals can be monitored for estrogen yield and the source of the chemical species can be traced back to an individual animal which is a significant advantage for quality control purposes. By identifying animals which are high producers the yield of a herd can be increased. This is not achievable in such a convenient way if the estrogen is extracted on a bulk basis in a large vat rather than individually on each mare.

Another advantage of this invention when a flow through collection system is used is that urine is disposed of in the paddock and does not present the waste disposal problem associated with treating urine in a central farm based system.

Because the collection system is based on individual animals the system can be used by small farms or studs without the need of a central farm based urine processing facility.

Those skilled in the art will realize that the present invention may be carried out in a number of embodiments Including arrangements not described herein. The system is also applicable to farmed animals other than horses and is applicable to cattle.

1. A method of extracting desired chemical species from pregnant animals urine which includes the steps of contacting the urine with dispersed particles of adsorber materials of a particle size above 200 microns and subsequently removing the species by washing the adsorber materials in a buffered aqueous solution of pH between 7.5 and 9.5.

2. A method as claimed in claim 1 in which the chemical species to be targeted are oestrogens, conjugated estrogens, equine chorionic gonadotropin, follicle stimulant hormone.

3. A method as claimed in claim 2 in which anionic exchange resin beads are used as the adsorber material.

4. A method as claimed in claim 2 in which the urine is filtered prior contact with the adsorber material.

5. Apparatus for the collection of valuable chemical species from pregnant animals urine which consists of a urine holding vessel suspended beneath the belly of the animal and a urine collection device attached to the perineum of the pregnant animal and communicating with the holding vessel in which the urine collection device and/or the holding vessel incorporates a removable urine permeable container containing an adsorbent material for said chemical species.

6. Apparatus for the collection of valuable chemical species from pregnant animals urine which consists of a urine holding vessel suspended beneath the belly of the mare and a urine collection device attached to the perineum of the mare and communicating with the holding vessel in which the urine holding vessel contains an organic water immiscible solvent for the chemical species.

7. Apparatus as claimed in claim 5 in which a filter is incorporated into the urine collection device upstream of the adsorber container.

8. Apparatus as claimed in claim 7 in which a funnel shaped polyamide mesh filter is incorporated in the urine collection device adjacent the mare's perineum.

9. Apparatus as claimed in claim 7 in which the filter is dosed with amucolytic agent to remove mucous and mucosal cells and reduce viscosity.

10. Apparatus as claimed in claim 6 in which the solvent is hexanol and the chemical species is estrogen or conjugated estrogens.

11. Apparatus as claimed in claim 5 in which the adsorbent material is an anionic resin or mixture of anionic resins in bead form.
